(19) United States
(12) Patent Application Publication
(10) Pub. No.: US 2002/0006612 A1
(43) Pub. Date: Jan. 17, 2002

(54) METHODS AND SYSTEMS OF IDENTIFYING EXCEPTIONAL DATA PATTERNS

(75) Inventors: LARRY D. GRELLER, COLLEGEVILLE, PA (US); FRANK L. TOBIN, BROOMALL, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(73) Assignee: Glaxosmithkline Corporate Intellectual Property, King of Prussia, PA (US)

(21) Appl. No.: 09/084,110

(22) Filed: May 21, 1998

(51) Int. Cl. C12Q 1/68

(52) U.S. Cl. 435/6

(57) ABSTRACT

A computational method for the identification of exceptional values in arrays of many sorts of intensity data is provided. The method is indifferent as to whether the intensities are experimental or computationally derived. Identification of patterns of selective expression of mRNA or protein gene products can be provided by the method of the invention.

### FIELD OF THE INVENTION

This invention relates to computer-based methods and systems for identification of exceptional patterns in data, such as selectively expressed genes and gene products.

### BACKGROUND OF THE INVENTION

The general problem of identifying exceptional patterns in data from many different sources can be viewed as an outlier identification problem. The outlier concept and statistical methods for outlier detection have an extensive literature [17-20]. Yet, what kinds of interpretations and quantitative treatments of data define an outlier remains fluid statistically and scientifically [17-20] and subjective [17].

Outlier detection problems arise in many different contexts. In the drug discovery field, intensity patterns may come from any array of intensity data derived from, for example, EST sequencing, microarray DNA hybridization, macromolecular gridding, compound assay data, molecular screening data, patient diagnostic and toxicological data. The conjunction of large-scale biology technologies, such as genomic sequencing or proteomics, and the need for new drug discovery targets has resulted in a need for more robust methods for detecting unusual expression patterns across many data sources. Thus, a need exists for useful quantitative objectivity to be brought to bear on the fundamental subjectivity of outlier detection.

### SUMMARY OF THE INVENTION

Accordingly, one aspect of the present invention is a method of identifying selectively expressed (exceptional) values in intensity data comprising analyzing statistical discordancy and gap criterion in a decision function wherein the decision function provides an overall confidence of above- or below-baseline exceptional intensity identification.

Another aspect of the invention is a method of identifying selectively expressed values in intensity data comprising:

(a) selecting intensity values from intensity data sources, wherein confidence in source quality exceeds a predetermined minimum threshold;

(b) determining if the number of selected intensities exceeds a predetermined minimum;

(c) applying a statistical discordancy test to identify statistically significant exceptional intensity values;

(d) determining a gap between the largest and another intensity by applying a minimum intensity gap criterion to the results of the statistical discordancy test;

(e) applying a decision function to the discordancy statistical significance and the gap to determine an overall confidence of exceptional intensity;

(f) identifying the degree of overall confidence of exceptional intensity; and

(g) displaying the results of step (f) on an output device.

Another aspect of the invention is a method of detecting selective expression of gene or gene products comprising:

(a) selecting intensity values from gene product data sources, wherein the source quality weight exceeds a predetermined minimum threshold;

(b) determining if the number of selected intensity values exceeds a predetermined minimum;

(c) applying a statistical discordancy test to identify statistically significant exceptional intensity values;

(d) determining a gap by applying a minimum intensity gap criterion to the results of the statistical discordancy test;

(e) applying a decision function to the statistical significance and the gap to determine an overall confidence of selective expression;

(f) identifying the degree of overall confidence of selective expression; and

(g) displaying the results of step (f) on an output device.

Yet another aspect of the invention is computer systems and computer readable media for performing the methods of the invention.

### BRIEF DESCRIPTION OF THE DRAWINGS

FIG. 1 diagrams simple stereotypical examples of selective expression types “up,” “down,” and “mixed”. Intensities vs. sources from a source set are plotted in arbitrary order. Selectively expressed intensities are indicated by encircled symbols.

FIG. 2 shows separation of a largest value from the n−1 others where xᵢ represents the intensities being compared in ascending order,xᵢ₋₁≦xᵢ, i=1, . . . , n. The basic measures for the Dixon test, namely the distance between the largest and the next-to-largest values (gap=xₙ−xₙ₋₁) and the distance between the largest and smallest values (xₙ−x₁), are used to calculate the separation ratio τ=gap/(xₙ−x₁).

FIG. 3 shows discordancy statistical significance adjusted for baseline position. Synthetic intensity data vs. source for a variety of different baseline levels of intensity, {0.25, 0.5, 0.75, and 0.9} are plotted.

FIG. 4 shows how erosion of statistical confidence increases as the baseline position increases towards the allowed maximum. Erosion of statistical confidence, i.e., loss of discordancy significance from the traditional Dixon value, is plotted vs. baseline encroaching toward the allowed maximum.

FIG. 5 shows a plot of a decision function, d, contours for selective expression (s.e.) overall confidence.

FIG. 6, panels A and B, shows examples of synthetic intensity (abundances) vs. source (library) data for assemblies. Panel C shows source qualities.

FIG. 7 shows stereotypical examples of selective expression in real data detected by the algorithm of the invention.

### DETAILED DESCRIPTION OF THE INVENTION

The method of the invention presents robust computational algorithms that identify exceptional values in intensity data. The algorithms are well-suited for the identification of exceptional values in many sorts of intensity data, even noisy data.

The method is generally applicable to any kind of intensity data where a distinguishable data source such as tissue, cDNA library, human, non-human (such as animal, plant, viral, bacterial or other microbial) source can be associated with each intensity value (e.g., gene or protein abundance, clone, biological or chemical activity, binding strength or genetic polymorphism assessment). For example, intensity values can be obtained from genomic sequencing, EST sequencing, microarray DNA hybridization, macromolecular gridding, compound assays, molecular screening assays, patient diagnostic or toxicological data sources. Assessments of trust, reliabilities, or relevances in the sources can be used as a basis for confidence. The intensities can be experimentally determined values, computationally derived values (e.g., abundances from cDNA data), or combinations. The method is indifferent to the experimental or computational lineages of the data to be analyzed. All that is required are triples of associated elements: entity (e.g., gene, protein, clone, assay, compound, etc.), intensity, and source. Table 1 lists some exemplary contexts where the method of the invention can be applied.

TABLE 1
Different Contexts for Application of the
Selective Expression Algorithm

| | | | | Typical Question |
|---|---|---|---|---|
| | | Source Set | In- | Associated with the |
| | Entity | comprising . . . | tensity | Context |
| 1 | contig, orf, | any patient, | abun- | What genes are |
| | assembly, | tissues or libraries | dance | selectively expressed? |
| | gene, clone | of interest generally | | |
| | or protein | | | |
| 2 | assembly, | different patients, | abun- | What selectively |
| | gene, or | tissues, libraries | dance | expressed genes are |
| | protein | receiving different | | associated with which |
| | | treatments | | tissues and specific |
| | | | | treatments? |
| 3 | assembly, | same tissue type | abun- | What are the selective |
| | gene, or | exposed to different | dance | expression dose |
| | protein | doses of a com- | | responses? |
| | | pounds | | |
| 4 | assembly, | same tissue type | abun- | What is the selective |
| | gene, or | receiving a series | dance | expression in response |
| | protein | of related | | to a specific series |
| | | treatments | | of treatments? |
| 5 | assembly, | same tissue type at | abun- | What is the kinetics |
| | gene, or | different times | dance | (i.e., time course) of |
| | protein | after a single | | selective expression? |
| | | treatment | | |
| 6 | assay | compounds | bio- | Is there an assay |
| | | | logical | whose intensity is |
| | | | or | selectively expressed |
| | | | chem- | among the compounds |
| | | | ical | tested? |
| | | | activity | |
| 7 | compound | genes of | abun- | Is there a gene of |
| | | toxicological | dance | toxicological interest |
| | | interest in a single | | selectively expressed |
| | | tissue, e.g., liver | | in response to the |
| | | | | compound? |
| 8 | compound | screening assays, | assay | Is there a selectively |
| | | chemical or | activity | expressed assay |
| | | biological | | activity in a |
| | | | | particular assay in |
| | | | | response to |
| | | | | compound? |
| 9 | patient, | any interpretable | abun- | What entities are |
| | assembly, | combination of the | dances | selectively expressed |
| | gene, | above | or assay | in any interpretable |
| | protein, or | | activ- | source set? |
| | compound | | ities | |
| | | | scored | |
| | | | for | |
| | | | compar- | |
| | | | ison | |

As used herein, “source” means any entity which may provide an intensity, e.g., tissue or EST library for genes or gene products, biological or chemical assay for compounds. “Genes” includes genomic DNA copy number, RNA, RNA transcripts. “Gene products” include proteins and RNA transcripts. If a source is experimentally manipulated or edited in any way, e.g., a normalized or subtracted cDNA library [9-11], it should not be included in the analysis lest its pattern of expressed genes be artificially skewed. This exclusion principle can be relaxed if all the sources being compared have been manipulated in the same way.

As used herein, “source set” means any collection comprising selected sources which may be analyzed for intensity patterns.

As used herein, the term “source confidence” represents the quality, the trust, the reliability, the knowledge of error, or the relative importance that can be attributed to the intensities obtained from the source. For example, a cDNA library sequenced in depth is a more reliable source than the same library sequenced to less depth.

As used herein, “source quality weights” represents quantitation of source confidences. Any consistent source quality weighting scheme can be used, but care must be exercised. If the weights are not faithful to the scientific reliabilities of the sources, any results dependent upon them can be improperly distorted. An edited or normalized cDNA library, for example, should be considered a low confidence source, i.e., given small weight, in a selective expression determination unless all the sources in the source set have been manipulated equivalently.

As used herein, “intensity” means a measured or calculated non-negative numerical value which is assigned to an observation, whether the observation is experimentally and/or computationally derived from data. For example, intensity could be a drug's binding affinity, a compound's activity in a screen, or a gene's abundance such as the gene product's copy number (molecules or concentration of mRNA) or amount of protein expressed. Intensity can be either an experimentally measured quantity, or less directly, a quantity which is calculated, for example, from analyses of cDNA assemblies [9, 12, 13]. For each source, the intensities may be scaled by a suitable norm, e.g., the maximum intensity, observed in that source. This is done to make intensities commensurably comparable from source to source, which is necessary if intensity patterns across sources are to be identified.

As used herein, a “discordant” observation is one that is “. . . statistically unreasonbable [or extreme] on the basis of some prescribed probability model.” [17]

As used herein, “exceptional” means a quantity that is markedly different from the other quantities against which it is compared.

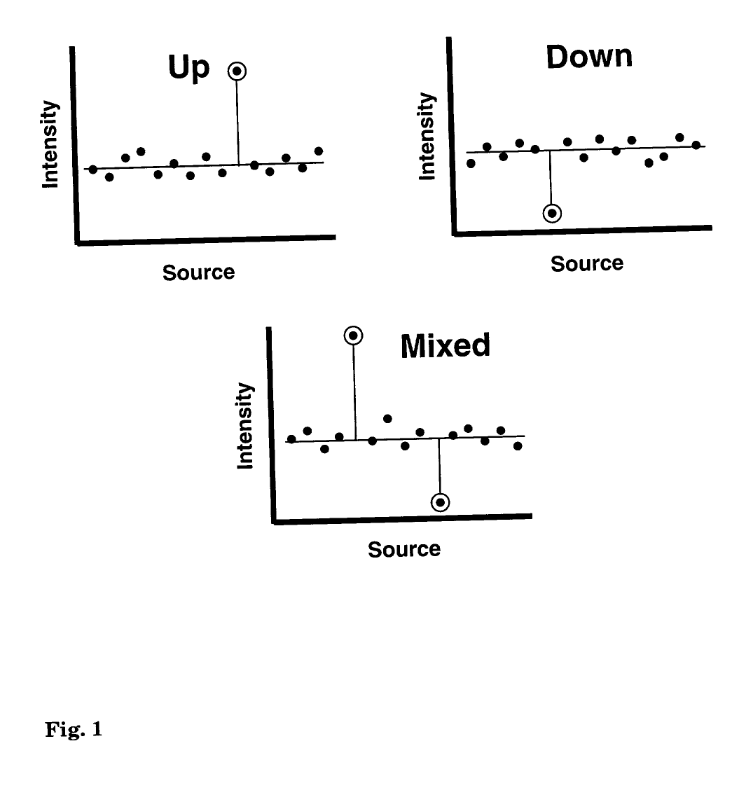

As used herein, “selective expression” is defined as a pattern among a collection of intensities in which there is an intensity which is markedly elevated, or markedly depressed, against a baseline level of intensity characteristic of the collection of intensities being compared. Hence, a “selectively expressed” intensity is an exceptional intensity. In particular, selective expression is a pattern in which there is a marked difference of intensity in a single source from a baseline level of expression established by the gene's or the entity's intensities in a source set. See FIG. 1 for stereotypical examples. The method of the invention does not require, however, that comparisons be made against all known sources. Instead, a carefully chosen subset of the known sources can be considered, especially since selective expression is a relative, not an absolute, assessment. Choice of source set enables the scientific context for expression comparisons to be tailored to the scientific questions being asked: organ systems vs. one another, tissues vs. one another (e.g., endothelium vs. smooth muscle or fibroblast), drug dose responses vs. one another, human vs. non-human species, chemical assays v. one another, etc.

A particular application of the invention provides a method that robustly identifies genes or proteins that are selectively expressed. The method combines assessments of the reliability of expression quantitation with a statistical test of intensity patterns. The method is applicable to small studies or to data mining of abundance data from large expression databases, whether mRNA or protein. The algorithm uniquely combines together a statistical test of discordancy, adjustments for baseline levels of the intensities (where baselines can be determined by source quality weighted averages), and adjustments for the separation of the largest and another intensity (gap) to give an overall assessment of confidence in selective expression. The algorithm achieves this by combining defined values—baseline adjusted discordancy and gap —into a decision function.

The algorithm is generally applicable to small- or large-scale expression-like data whether derived from DNA sequencing, proteomics, compound assays, pharmacogenomics, or toxicological safety assement, etc. The method can be implemented as computer programs that analyze databases of gene abundances on a regular basis.

The method is particularly useful in identifying biologically and pharmacologically interesting selectively expressed genes, hence, having objective implications for further analysis. It is well-established that DNA sequence copy number and mRNA levels in eukaryotic cells are present in a variety of abundance classes [1-3]. Very wide differences in gene expression level, i.e., in intracellular mRNA copy number, abundance, or in amount of gene product, are possible within the same cell. For example, it has been estimated that the copy numbers of expressed genes can vary from 1 to about 200,000 [4].

Further, the same cell type, as well as different cell types, may exhibit different patterns of gene expression when exposed to different conditions [5, 8]. Assessing differences in expression patterns, therefore, can be used to gauge differences in cell physiology and tissue behavior, intrinsically or in response to many different kinds of stimuli. As these differences may be correlated with fundamental biological phenomena or disease processes, delineations of patterns of gene or protein expression among normal and diseased states or patients exposed to drugs are of increasing importance in medical diagnostics and therapy.

Two stereotypical simple selective expression situations are possible: “up,” where expression is significantly elevated in a specific tissue when compared against the baseline level in the other tissues; “down,” where the expression in a specific tissue is significantly depressed when compared against the baseline expression in the other tissues. “Up” selective expression may be an important indication that the gene has been specifically activated, up-regulated, or its product differentially elevated in association with certain phenomena or agents affecting a particular tissue's biology. Similarly, “down” selective expression is either a significant down-regulation or essentially an inactivation of the gene (e.g., tumor suppressor loss of function) in association with specific biological events. Such broad phenomena as morphogenesis, differentiation, metabolic alteration, mutagenesis, bacterial and viral infection, physiological stress, disease, drugs and therapeutic interventions, etc., can manifest or cause selective expression effects.

For example, the method of the invention can compare relative levels of mRNA transcripts or relative levels of protein products. Despite the inherent difficulties in precisely measuring which mRNA species are translated and in what relative proportions, reliable enough information on expression levels can be obtained [5, 11, 14]. Moreover, the established experimental techniques of cDNA and EST sequencing, especially when employed on a large scale, can provide ESTs that can be combined computationally into assemblies [9]. Assemblies can be interpreted as putative expressed genes, though to widely varying levels of confidence in the assignments of assemblies to genes [12, 13]. Abundances of expressed genes or assemblies obtained from sampling are dependent upon the depth of the sampling [15, 16] and contribute to inaccuracies in the computed intensities [13].

In one embodiment, the invention provides a computational method (algorithm) of identifying selectively expressed values in intensity data comprising analyzing statistical discordancy and gap criterion in a decision function wherein the decision function provides an overall confidence of above- or below-baseline exceptional intensity identification. The statistical discordancy can be adjusted for baseline intensity levels.

In an alternate embodiment, the invention provides a method of identifying exceptional values in intensity data comprising:

(a) selecting intensity values from intensity data sources, wherein confidence in source quality exceeds a predetermined minimum threshold;

(b) determining if the number of selected intensities exceeds a predetermined minimum;

(c) applying a statistical discordancy test to identify statistically significant exceptional intensity values;

(d) determining a gap between the largest and another intensity by applying a minimum intensity gap criterion to the results of the statistical discordancy test;

(e) applying a decision function to the discordancy statistical significance and the gap to determine an overall confidence of exceptional intensity;

(f) identifying the degree of overall confidence of exceptional intensity; and

(g) displaying the results of step (f) on an output device.

In another embodiment, the invention provides a method of detecting selective expression of genes or gene products comprising:

(a) selecting intensity values from gene product data sources, wherein confidence in source quality exceeds a predetermined minimum threshold;

(b) determining if the number of selected intensities exceeds a predetermined minimum;

(c) applying a statistical discordancy test to identify statistically significant exceptional intensity values;

(d) determining a gap between the largest and another intensity by applying a minimum intensity gap criterion to the results of the statistical discordancy test;

(e) applying a decision function to the discordancy statistical significance and the gap to determine an overall confidence of selective expression;

(f) identifying the degree of overall confidence of selective expression; and

(g) displaying the results of step (f) on an output device.

In these embodiments, the statistical discordancy test results of step (c) can be adjusted according to the difference between a baseline position and a maximum allowed intensity to achieve a baseline adjusted statistical significance. Preferably, the gap is determined between the largest and the next-to largest intensity. Further, when available, source quality confidence is based on trust, reliability, knowledge of error or relevance. Preferably, the intensity baseline position is determined by a source quality weighted average of the intensities.

In addition to display on an output device such as a monitor or a printer, the identity of the selectively expressed gene products can be stored in a database. The methods of the invention can further comprise the step of characterizing the selectively expressed gene product. Characterization can be done on the basis of of sequence, structure, biological function or other related characteristics. Once categorized, the database can be expanded with information linked to biological function, structure or other characteristics. Further, selectively expressed genes or gene products can be characterized on the basis of expert commentary from relevant human specialists or by the results of biological experiments. If desired, the selectively expressed entites detected by the method may be confirmed experimentally by techniques well known to those skilled in the art [2, 5-7].

In step (a), minimum source quality weight criterion are applied. For an entity's collection of intensities to be analyzed from the source set (e.g., a particular gene's abundances in a source set of libraries), intensities are selected from only those sources whose corresponding quality weight (i.e., trust, reliability, or relevance) exceeds a minimum. Minimum quality thresholds can be determined by those skilled in the art by applying scientific judgments concerning the reliabilities or relevances of the sources. Oftentimes as data is being accumulated, a source's quality will change with the data, requiring the selective expression algorithm to be re-applied. Source quality weighting is considered optional, in which case this is equivalent to either no weighting or all weights being the same, e.g., unity.

Step (b) determines whether the number of selected intensity values exceeds a predetermined minimum. In sub-step (b1), there is the option of whether or not zero intensities in the source set are considered or ignored. If the option of ignoring, hence omitting, zero intensities is taken, then sub-step (b2) determines whether or not a non-zero intensity exceeds its source's detection limit (experimentally or computationally). In sub-step (b2) if a non-zero intensity does not exceed its source's detection limit, then that intensity is considered equivalent to zero and therefore omitted as in sub-step (b1). For an entity being analyzed for selective expression (e.g., a particular gene in a source set of libraries), if there is at least a predetermined minimum number of intensities surviving this step and that exceeds appropriate detection limits (discussed below), this entity (e.g., gene) and these intensities are marked for further analysis. In general, the minimum number of intensities will be enough to make confident identifications of exceptional intensities. However, a lesser number can be used with the understanding that the confidences in the assessments will be lower [17]. The minimum number of intensities is 3. Most preferably, the minimum number of intensities will be at least 10.

With respect to intensity detection limits, if an intensity appears to be absent from a particular source, then either (1) the intensity is actually not expressed in the source, or (2) the intensity is indeed expressed in the source but is smaller than the minimum intensity which can be measured, the detection limit. In case (2), since the intensity is not truly absent but instead occurs below the detection limit, it is thus recorded as absent. In the method of the invention, absent intensities can be considered as genuine absence only for very high quality sources with very low detection limits. All absent or sub-detection limit intensities are therefore ignored. However, the method does not require adopting this philosophy.

Step (c) applies a statistical discordancy test to identify statistically significant exceptional intensity values. Statistical tests of discordancy are known to those skilled in the art [17-20]. The resulting statistical significance is used to score how exceptional the putative discordant intensity is. The test is applicable to exceptionally small intensities (“down” selective expression) as well as exceptionally large intensities (“up” selective expression).

A uniform distribution Dixon test [17] can be used in the method of the invention for the statistical test of discordancy. A uniform distribution assumes only that intensities are finite and there is no a priori most probable intensity. This is a reasonable parsimonious choice for an actually unknown inter-source intensity distribution; it is a choice which confers a priori only a very weak bias in distribution shape or in central tendency.

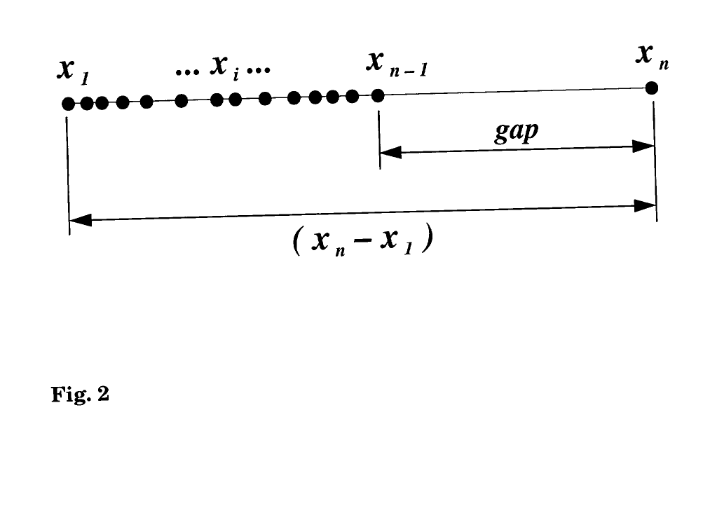

The first graph in FIG. 1 diagrammatically shows a source set of intensities having a single exceptionally large intensity. Such data can be sorted in ascending order and re-plotted as in FIG. 2. When values are sorted, the relative separation between the largest value and the remaining values becomes clearer. The size of the gap between the largest and next largest value divided by the distance between the largest and smallest values (see FIG. 2) is an obvious measure of the separation of the largest value from all the other values. This “separation ratio” (equation 4 below) is the core of the statistic employed in the Dixon test for a single largest discordant value among uniform samples [17]. It captures the logical underpinnings of the statistical test.

In the case of the more general m^{th} largest discordant value Dixon test, the appropriate changes in the formulas for the degrees of freedom and the separation ratio dependent statistic [17] can be employed. The more general case is applicable to the problem of simultaneously identifying more than one selectively expressed intensity in a collection of intensities. For application of the test to selective expression, it was found that the single largest value test was sufficient and is preferred. The mathematical details follow.

For a selected entity (e.g., gene), let the vector f′ comprise the entity's intensities from the n different sources of the source set which are to be analyzed after step (b). Let q be the vector comprising the corresponding source quality weights. If source quality weights are not assigned, the elements of q are set to unity. The elements of f′ and q are real numbers >0. The sequential order of the vectors′ elements is arbitrary since the order of the sources in the source set can be arbitrary. However, once an order of sources is chosen, the elements of f and elements of q must appear in the same order since the respective correspondences between qualities and sources must be maintained.

Essentially the same method that is used for the identification of exceptionally large intensities, i.e., “up” selective expression, can be employed with minor modifications for the identification of exceptionally small intensities, i.e., “down” selective expression. Define vectors f and f_{down} from f′ as follows:

$$\{ \begin{matrix}{f}_{max} = maximum ( {f}^{′} ) & \\ f = {f}^{′} / {f}_{max} , & “ up ” selective expression \\ {f}_{down} = 1 - {f}^{′} / {f}_{max} , & “ down ” selective expression\end{matrix} \qquad ( 1 )$$

Though the mathematical form of the method is unchanged by using f_{down} in place of f, identifying exceptionally small values is fundamentally, and practically, different from identifying exceptionally large values. This is because there can be intensities in f that are so minute (though still above a very small detection limit) as to be measurements indistinguishable from noise, making them useless as reliable values in a discordancy test. One way to remedy this difficulty is to restrict f to comprise only those values that are considerably larger than the detection limit. However, once equation 1 is used, the same baseline adjustment technique used for f (step (d)) can be applied to f_{down}.

Define x as the vector that comprises the n elements of f sorted in ascending order, i.e., xᵢ₋₁≦xᵢ. Next, compute the Dixon critical statistic T_{critical} from the elements of x (equations 3 through 5 below). Then use the Dixon test (equation 2 below) to compute the discordancy significance probability of the largest intensity among these intensities being compared. According to the Dixon test for a single largest outlier [17], the significance probability sp that the largest sample is discordant, i.e., exceptionally large, is given by

$$\begin{matrix}sp = Probability [ t \geq {T}_{critical} ] \\ = 1 - {\int }_{0}^{{T}_{critical}} {F}_{2 , n - 2} ( z ) ⅆ z\end{matrix} \qquad ( 2 )$$

where t is a dummy variable which represents any possible value of (n−2)τ/(1−τ) for fixed n, F is the standard statistical F-distribution with degrees of freedom 2 and (n−2) [21], and where

gap=xₙ−xₙ₋₁ (3)

τ=gap/(xₙ−x₁) (the separation ratio), (4)

T_{critical}=(n−2)τ/(1−τ). (5)

The interpretation of significance probability, sp,is the natural one: the smaller the significance probability, the more exceptionally large is the largest value, xₙ, when compared against all the other values of x. The significance probability given by the fundamental equation (2) can be reduced algebraically [17] to the very simple form

log₁₀(sp)=(n−2)log₁₀(1−τ). (6)

Equation 6 conveniently quantitates the theoretical statistical significance that the largest sample is exceptionally large. From equation 6, the significance probability decreases markedly as the separation ratio τ approaches 1. Moreover, this effect is stronger, the larger the sample size n. For a fixed sample separation ratio τ, the logarithm of the significance probability decreases linearly with the number of samples n since τ<1 (equation 6).

Note that the conventional Dixon definition of the separation ratio τ effectively normalizes the separation between the largest and next-to-largest intensities by the range spanned by all the intensities being compared. This is what confers an apparent dynamic range indifference to the Dixon test. However, the effective dynamic range of the analyzed intensities with respect to a maximum allowed intensity is important to the method of the invention. The mathematical details of the adjustment made to the Dixon test to remedy the test's otherwise indifference to dynamic range is discussed in the step (d) details below.

Note that it can be shown numerically and analytically that

$$Δ log ( sp ) \approx \frac{\partial log ( sp )}{\partial τ} Δ τ \approx \frac{\partial log ( sp )}{\partial τ} Δ ( ( {x}_{n} - {x}_{n - 1} ) / ( {x}_{n} - {x}_{1} ) )$$

is small for small changes in gap or in any of x₁, xₙ₋₁, or xₙ. This obviates replacing any of x₁, xₙ₋₁, or xₙ by respective source quality weighted estimates in the computation of τ in equation 4 above. However, a role for q persist in step (d). In step (d), the statistical discordancy test results are adjusted according to the difference between a baseline position and a maximum allowed intensity to achieve a baseline adjusted statistical significance. The baseline position can be determined by a source quality weighted average of the intensities. Apart from the putative discordant intensity, the other intensities among those being compared can be characterized as being clustered about a baseline level. The statistical test of discordancy results from step (c) are adjusted according to the difference between the baseline position and the maximum allowed intensity. The adjustment to the statistical significance is to increasingly downgrade it as the baseline becomes closer to the maximum allowed intensity. The baseline dependent adjustment is based on the dynamic range of the values being increasingly compressed, hence less mutually distinguishable, the closer the baseline is to the allowed upper limit. But, the Dixon test is indifferent to dynamic range compression, as noted above. However, since the discrimination of values is necessarily eroded as the effective dynamic range is compressed, the confidence in outlier detection (discordancy) should be eroded correspondingly. The mathematical details are explained below.

The position of the baseline, i.e., a level which characterizes the non-extreme values of a collection of intensities, should affect the confidence of the selective expression determination as described above. Along these lines, if the dynamic range is compressed in the extreme, then the measurements would all become essentially indistinguishable since the accuracy of real measurements is always limited. Hence, discordancy detection would be meaningless in such a situation, regardless of how discordancy is computed, since separations between the values involved would be indistinguishable from numerical or measurement noise. However, the Dixon test is indifferent to the dynamic range of the data, as noted in step (c). This phenomenon of indifference to dynamic range is not idiosyncratic to Dixon tests, but is inherent generally to any excess/spread, range/spread, or deviation/spread discordancy statistical test [17]. So, even if the dynamic range is compressed, as long as the difference between the largest and the next-to-largest values is proportionally compressed, the traditional Dixon test significance is unchanged. Thus, the traditional Dixon test must be modified to correct for erosion in confidence in discordancy detection as a compression in dynamic range occurs.

To accomplish this, the Dixon significance is adjusted by a baseline adjustment factor λ. λ ε (0,1) is designed to attenuate the traditional Dixon separation ratio τ (equation 4) so that the adjusted τ is

τ_{adjusted}=λτ. (7)

We choose λ to be a sigmoidal function of baseline with the parameters of the sigmoid chosen so that λ remains approximately unity until the baseline encroaches substantially on the maximum allowed intensity, e.g., typically 1. For example,

$$λ = {( 1 + {( \frac{{\hat{x}}_{baseline}}{c} )}^{b} )}^{- 1} \qquad ( 8 )$$

where c is the value of \hat{x}_{baseline} for which λ=0.5, i.e., the sigmoid's point of inflection, and b>0 controls the steepness of λ decay with increasing \hat{x}_{baseline}. In practice, we typically use c=0.8 and b=10 in equation 8. \hat{x}_{baseline} is a source quality weighted estimator of x baseline, which excludes the putative extreme value xₙ, e.g., a weighted average

$${\hat{x}}_{baseline} = {\sum }_{i = 1}^{k} {q}_{i} {x}_{i} / {\sum }_{i = 1}^{k} {q}_{i} \qquad ( 9 )$$

In equation 9, k<n to insulate the baseline estimate from possible undue influence of a putative extreme value xₙ. Though we prefer quality weighted baseline estimates, one can choose to ignore quality differences in \hat{x}_{baseline}, and therefore, substitute unity for the qᵢ. In which case, equation 9 becomes the simple average.

For this τ adjustment for baseline concept, any function can be chosen which has the effect of substantially diminishing outlier significance when baselines encroach upon the maximum allowed intensity. We find sigmoids to be especially convenient. Thus, the traditional Dixon outlier significance probability (equation 6) is adjusted for the baseline by the simple formula:

log(sp_{adjusted})=(n−2)log(1−τ_{adjusted}) (10)

where τ_{adjusted}=λτ, λ is computed from equations 7 and 8.

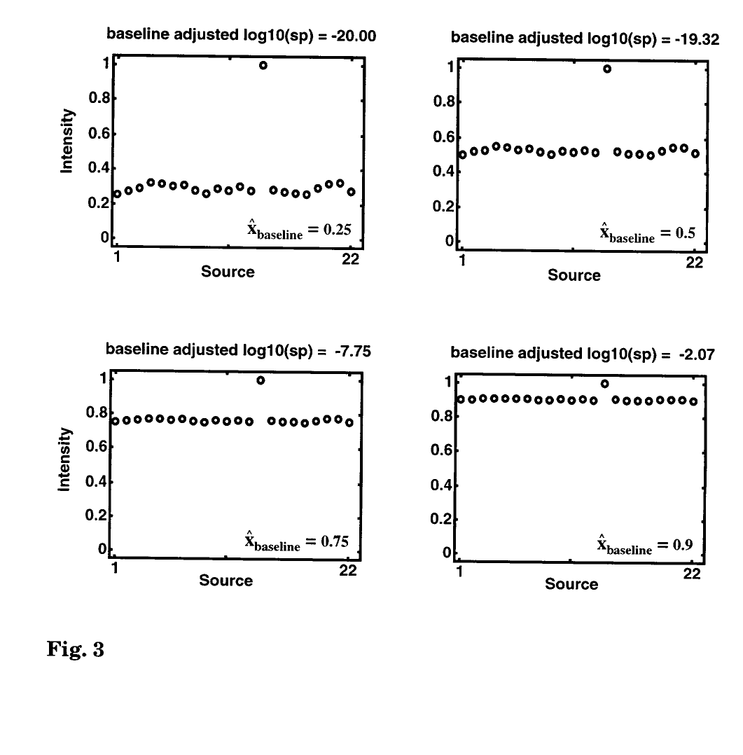

To illustrate, consider the examples in FIG. 3 and the corresponding Table 2. Each row in Table 2 represents a different, yet related, set of intensities. x denotes the vector comprising a set of intensities sorted in ascending order. In each example and throughout the calculations, the source set size is held constant at n=22, and the maximum intensity xₙ is held constant at 1. However, for each example (row) the minimum intensity x₁ is set to the value in the first column. For illustrative simplicity, x₁ is also taken to be the baseline estimate \hat{x}_{baseline} since the non-extreme values are so narrowly clustered near x₁ in these examples. Quality weights are not needed, then, in these simplified baseline estimates.

TABLE 2
Affect of Baseline Position on the Adjusted
Dixon Statistical Significance Probability

| Base- | | | | τ_{adjusted} = | | |
|---|---|---|---|---|---|---|
| line | χₙ₋₁ | gap | λ | λτ | log₁₀(sp_{adj.}) | Δlog₁₀(sp) |
| 0.25 | 0.32 | 0.68 | 1.00 | 0.90 | −20.00 | 0.00 |
| 0.50 | 0.55 | 0.45 | 0.99 | 0.89 | −19.32 | 0.68 |
| 0.75 | 0.78 | 0.22 | 0.66 | 0.59 | −7.75 | 12.25 |
| 0.90 | 0.91 | 0.09 | 0.24 | 0.21 | −2.07 | 17.93 |

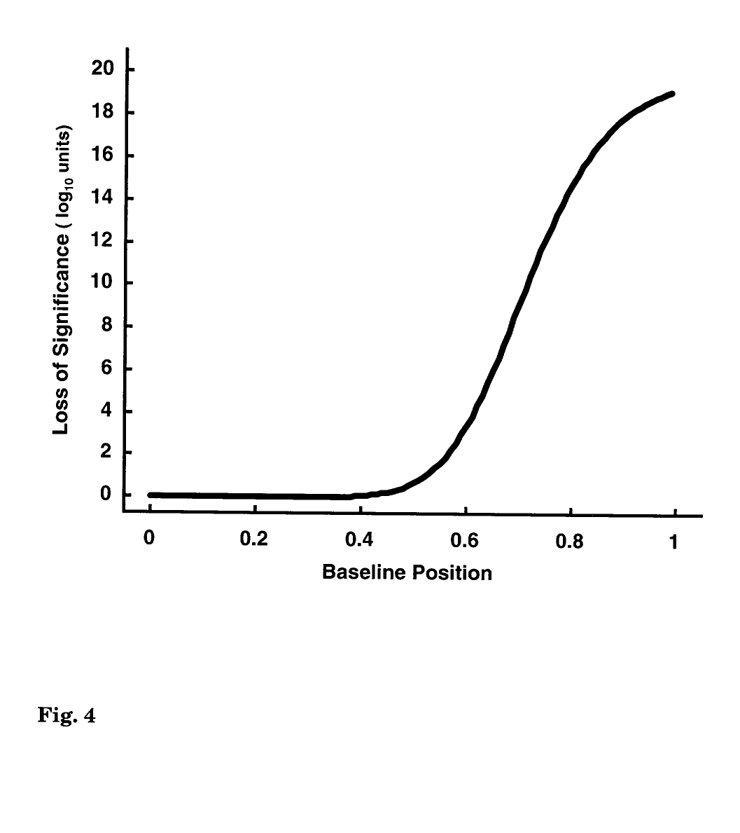

Each example set of synthetic intensity values corresponding to \hat{x}_{baseline} values {0.25, 0.5, 0.75, 0.9} are plotted respectively in FIG. 3. In each case, the traditional Dixon significance probability (log₁₀(sp)=−20) is kept fixed. Constant Dixon significance, regardless of baseline position, is achieved deliberately in these synthetic data by adjusting the second-largest intensity (xₙ₋₁), shown in column 2, according to equations 3 and 7. Hence, the gap between the largest and next-to-largest intensities (xₙ−xₙ₋₁) necessarily decreases as the baseline increases; yet, the traditional Dixon significance remains unchanged. But, the closer the baseline is to the allowed maximum, (xₙ=1), the less confidence there is in an assessment of discordancy. Therefore, the statistical significance must be reduced from the traditional Dixon value according to how the baseline encroaches upon the allowed maximum. This is done by diminishing the separation ratio T according to a sigmoidal function of the baseline (equations 7 and 8). As can be seen, the baseline adjusted significance decreases as the baseline increases towards the allowed maximum The erosion of traditional Dixon significance increases as baselines are continuously increased towards the allowed maximum (FIG. 4). See also Table 2 where xₙ₋₁ (column 2) is computed by using equations 4, 5 and 6 to insure that the traditional Dixon discordancy significance probability remains fixed at log₁₀(sp)=−20 even though x₁ is different in each example. The baseline adjustment factor X computed using equation 8 with b=10 and c=0.8 is in column 4. The effect of the baseline adjustment factor λ on the traditional Dixon significance is shown in columns 5 and 7. The loss of statistical significance, Δlog₁₀(sp), between the baseline adjusted significance and the traditional significance in column 7 is in log₁₀ units. It is plotted as a continuous function of baseline in FIG. 4. As desired for baseline adjustments of statistical significance, the erosion in confidence reflected becomes substantial as the baseline encroaches upon an intensity upper limit.

An important general principle is illustrated by these examples: Though the traditional Dixon significance probability can remain apparently extremely significant (e.g., 10⁻²⁰) even as the dynamic range of the data is compressed ever smaller (represented here by the baseline coming ever closer to an allowed maximum), a baseline adjusted significance probability can nonetheless reflect the erosions of statistical significance that should occur in data whose dynamic range is substantially compressed.

It should be noted that while there is no intrinsic method to determine how much discordancy significance probability ought to be attenuated quantitatively as a function of baseline levels, scientific judgment of those skilled in the art concerning data accuracy, the resolving power of intensity measurement techniques, and the dynamic range of intensity data can be used to design significance adjustment functions. The role of scientific judgment in this situation is analogous to that for establishing source quality weighting and for subjectively interpreting discordancy.

In step (d), a gap is determined by applying a minimum intensity gap criterion to the results of the statistical discordancy test. The gap, i.e., the separation between the largest and the next-to-largest intensities, is a fundamental ingredient in discordancy assessment. See FIG. 2 and the description of step (c) above. If the gap is below or near the resolving power of the technique providing the intensity data, there is necessarily negligible confidence in the assessment of discordancy, regardless of how the discordancy statistical significance is computed. This is because a gap commensurable with the intensity measurement technique's resolving power means that the difference between the values constituting the gap is indistinguishable from measurement noise. Therefore, a minimum gap criterion should be applied in conjunction with the discordancy statistical test from step (c). While there is no objective formula for establishing the minimum gap criterion, scientific judgment of those skilled in the art can be used to set the minimum gap threshold which takes into account the accuracy and resolving power of the technique that provides the intensity data. The mathematical details of step (d) follow.

Those gaps which meet a minimum gap threshold gₜₕᵣₑₛₕ are rescaled linearly between gₜₕᵣₑₛₕ and the maximum allowed intensity xsup. Call these rescaled gaps g, e.g:

$$g = \{ \begin{matrix}0 , if gap \leq {g}_{thresh} \\ ( gap - {g}_{thresh} ) / ( 1 - {g}_{thresh} ) , if gap > {g}_{thresh}\end{matrix} \qquad ( 11 )$$

Analogously, linearly transform the baseline adjusted significance log₁₀(sp_{adjusted}) (equation 10) between the weakest-to-strongest statistical significance that one is willing to accept, i.e., between log₁₀(sp)ₜₕᵣₑₛₕ and log₁₀(sp)_{inf}, respectively. The lower bound log₁₀(sp)_{inf} is the statistical significance beyond which stronger statistical significance is essentially inconsequential. Denoting by s, (0≦s≦1), as this transformation gives:

$$s = \{ \begin{matrix}0 , if {log}_{10} ( {sp}_{adjusted} ) \geq {{log}_{10} ( sp )}_{thresh} \\ 1 , if {log}_{10} ( {sp}_{adjusted} ) \leq {{log}_{10} ( sp )}_{inf} \\ \frac{{log}_{10} ( {sp}_{adjusted} ) - {{log}_{10} ( sp )}_{thresh}}{{{log}_{10} ( sp )}_{inf} - {{log}_{10} ( sp )}_{thresh}} , if {{log}_{10} ( sp )}_{thresh} < {log}_{10} ( {sp}_{adjusted} ) < {{log}_{10} ( sp )}_{inf}\end{matrix} \qquad ( 12 )$$

Preferably, log₁₀(sp)ₜₕᵣₑₛₕ=−5. Less preferred is log₁₀(sp)ₜₕᵣₑₛₕ=−3. Preferably, log₁₀(sp)_{inf}=−20, which allows the adjusted significance probability a dynamic range of 10¹⁵.

In step (e), a decision function is applied to the baseline adjusted statistical significance and the gap to determine an overall confidence of selective expression. In step (f), the degree of overall confidence of selective expression is identified.

The gap from step (d) should be combined with the baseline adjusted statistical significance of discordancy from step (c) in order to provide an overall confidence of selective expression. This is accomplished by applying a decision function that is dependent upon both of these. The decision function d ranks the assessment into Low (weak), Medium (moderate), or High (strong) confidence of selective expression. But, if either a minimum baseline adjusted discordancy significance was not met or a minimum gap was not exceeded, that entity and its set of intensities is marked as not exhibiting selective expression. The construction and employment of a representative decision function is described below.

While there is no intrinsic method to determine the mathematical forms of decision functions, there is practical utility in assigning overall confidences to separate weak from strong predictions of selective expression. An interpretation of the strength of a result is often for setting priorities for further analyses of the data and new experiments.

Decision function d near 0 is interpreted as very weak overall confidence, while d near 1 is very strong overall confidence in selective expression. d is designed to capture the following notions of confidence:

| | | scaled gap g | scaled gap g |
|---|---|---|---|
| | | weak | strong |
| | | (g˜0) | (g˜1) |
| scaled sig. | weak | weak | strong |
| prob. s | (s˜0) | (d˜0) | (d˜1) |
| scaled sig. | strong | moderate | strong |
| prob. s | (s˜1) | (d˜1) | (d˜1) |

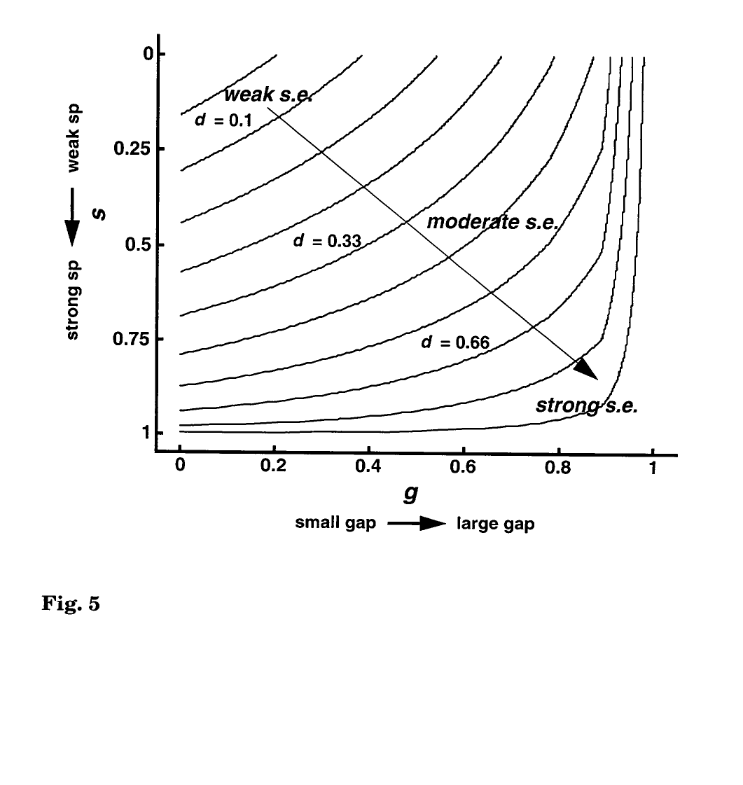

d is (1) strong when both the baseline adjusted log₁₀sp and the gap are strong (i.e., both s and g are near 1); (2) weak when both the log₁₀sp and the gap are weak (i.e. s and g near 0); (3) moderate when the log₁₀sp is strong but the gap is weak; (4) but strong nonetheless when the gap is strong yet the log₁₀sp is weak. Notions (3) and (4) make sense because both the log₁₀sp and the gap that are considered in the decision function confidence assessment are stronger their respective minimum thresholds. Either log₁₀sp or gap weaker than their respective minimum thresholds is not selective expression, and immediately d=0 in such cases. There is no a priori requirement that d be symmetrical with respect to g and s. In fact, in practice, an asymmetry is preferred that gives d near 1 for large gaps as long as log₁₀sp is stronger than a threshold value. Using these principles, a useful decision function is:

$$d ( g , s ) = 1 - {( [ {( 1 - s )}^{α} {( 1 - g )}^{β} {( \frac{δ ( 1 - g ) + ( 1 - δ ) ( 1 - s )}{( 1 - g ) + ( 1 - s )} )}^{γ} ] )}^{φ} \qquad ( 13 )$$

where α≧0, β≧0, λ≧0, and δ (0<δ<1) are independent parameters chosen empirically, and where φ is defined by φ=(α+β+γ)⁻¹. Observe that the term in brackets amounts to a numerical version of a logical AND of three terms, the third term of which amounting to a numerical logical OR of two terms blended in a proportion controlled by δ. Typically, we choose α=β=γ=1.5 and δ=0.3. FIG. 5 shows this decision function d plotted as a series of constant-d contours on (g,s)-space. (g,s) are the respective linear transformations of gap and baseline adjusted log₁₀(sp) between the weak thresholds and strong limits. See equations 11-13.

Step (f): Though there is no intrinsic method for setting break points between weak, moderate, and strong overall confidences, in practice the strength of the selective expression overall degree of confidence breakpoints for d are taken to be ⅓ and ⅔, respectively.

Another aspect of the invention is a computer system for identifying selectively expressed values in intensity data. A representative computer system includes a hardware environment on which the methods of the invention may be implemented. The hardware environment includes a central processing unit, a memory device, a display and a user interface device. An exemplary hardware environment is a Sun Microsystems Ultra 1 running a UNIX operating system, having a display and keyboard and/or mouse input devices.

In one embodiment, the computer system for identifying selectively expressed values in intensity data comprises means for analyzing statistical discordancy and gap criterion in a decision function wherein the decision function provides an overall confidence of above- or below-baseline exceptional intensity identification.

In another embodiment, the computer system for identifying exceptional values in intensity data comprises:

(a) means for selecting intensity values from intensity data sources, wherein confidence in source quality exceeds a predetermined minimum threshold;

(b) means for determining if the number of selected intensities exceeds a predetermined minimum;

(c) means for applying a statistical discordancy test to identify statistically significant exceptional intensity values;

(d) means for determining a gap between the largest and another intensity by applying a minimum intensity gap criterion to the results of the statistical discordancy test;

(e) means for applying a decision function to the discordancy statistical significance and the gap to determine an overall confidence of exceptional intensity;

(f) means for identifying the degree of overall confidence of exceptional intensity; and

(g) means for displaying the results of step (f) on an output device.

In another embodiment, the computer system comprises a central processing unit executing a selectively expressed value identifying program stored in a memory device accessed by the central processing unit; a display on which the central processing unit displays screens of the exceptional value identifying program in response to user inputs; and a user interface device.

Another aspect of the invention is a computer readable medium containing program instructions for identifying selectively expressed values in intensity data comprising analyzing statistical discordancy and gap criterion in a decision function wherein the decision function provides an overall confidence of above- or below-baseline exceptional intensity identification.

In another embodiment, the computer readable medium contains program instructions for identifying exceptional values in intensity data, the program instructions comprising:

(a) selecting intensity values from intensity data sources, wherein confidence in source quality exceeds a predetermined minimum threshold;

(b) determining if the number of selected intensities exceeds a predetermined minimum;

(c) applying a statistical discordancy test to identify statistically significant exceptional intensity values;

(d) determining a gap between the largest and another intensity by applying a minimum intensity gap criterion to the results of the statistical discordancy test;

(e) applying a decision function to the discordancy statistical significance and the gap to determine an overall confidence of exceptional intensity;

(f) identifying the degree of overall confidence of exceptional intensity; and

(g) displaying the results of step (f) on an output device.

The present invention will now be described with reference to the following specific, non-limiting examples.

### EXAMPLE 1

### Selective Expression Detection in Synthetic Data

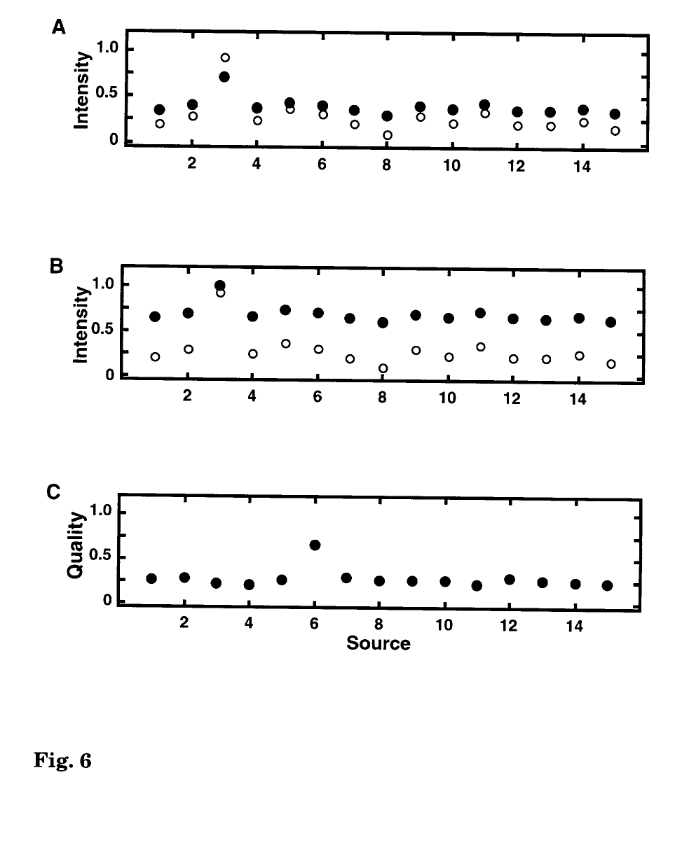

In FIG. 6, synthetic data representative of real assembly abundances are shown. Panel A shows Set 2 (filled circles) and Set 1 (open circles) for comparison; panel B shows Set 3 (filled circles) and Set 1 (open circles) for comparison. In panels A and B, the putative selective expression occurs in the third Source. Panel C shows the source qualities corresponding to the intensities.

The numerical values of the source qualities and corresponding intensity data are in Table 3. The computed numerical results using the method of the invention are summarized in Table 4. Though these intensity and source quality data are synthetic, they are representative of real data derived from a large abase of gene abundances and library qualities.

TABLE 3
Synthetic Intensity (Abundance) and Source
(Library Quality) Assembly Data

| Source | Quality | Example 1 | Example 2 | Example 3 |
|---|---|---|---|---|
| 1 | 0.26 | 0.19 | 0.35 | 0.64 |
| 2 | 0.27 | 0.29 | 0.39 | 0.68 |
| 3 | 0.22 | 0.92 | 0.71 | 1.00 |
| 4 | 0.20 | 0.24 | 0.37 | 0.66 |
| 5 | 0.26 | 0.37 | 0.43 | 0.72 |
| 6 | 0.65 | 0.31 | 0.40 | 0.69 |
| 7 | 0.29 | 0.21 | 0.35 | 0.64 |
| 8 | 0.26 | 0.10 | 0.30 | 0.59 |
| 9 | 0.26 | 0.30 | 0.40 | 0.69 |
| 10 | 0.26 | 0.23 | 0.37 | 0.65 |
| 11 | 0.21 | 0.35 | 0.43 | 0.72 |
| 12 | 0.28 | 0.22 | 0.36 | 0.65 |
| 13 | 0.26 | 0.21 | 0.36 | 0.64 |
| 14 | 0.25 | 0.26 | 0.38 | 0.67 |
| 15 | 0.22 | 0.17 | 0.34 | 0.63 |
| \hat{χ}_{baseline} | | 0.25 | 0.37 | 0.66 |
| gap | | 0.55 | 0.28 | 0.28 |
| τ | | 0.67 | 0.67 | 0.67 |
| no baseline | | | | |
| adjustment | | | | |
| τ_{adjust} | | 0.67 | 0.67 | 0.58 |
| baseline | | | | |
| adjusted | | | | |

TABLE 4
Application of Selective Expression Algorithm
to Synthetic Data

| | Base- | | | | | | |
|---|---|---|---|---|---|---|---|
| | line | | | | log₁₀ | | |
| Set | Adjust | λ | gap | τ | (sp) | d | Comments |
| 1a | no | | 0.55 | 0.67 | −6.26 | 0.33 | Reference example. |
| 1b | yes | 1.00 | 0.55 | 0.67 | −6.26 | 0.33 | Same as 1a; λ has no |
| | | | | | | | effect. |
| 2a | no | | 0.28 | 0.67 | −6.26 | 0.24 | d different from 1a due |
| | | | | | | | to gap only. |
| 2b | yes | 0.99 | 0.28 | 0.67 | −6.26 | 0.24 | d different from 1a due |
| | | | | | | | to gap; λ has no effect. |
| 3a | no | | 0.28 | 0.67 | −6.26 | 0.24 | d different from 1a due |
| | | | | | | | to gap only. |
| 3b | yes | 0.87 | 0.28 | 0.58 | −4.90 | 0.00 | d different from 1a due |
| | | | | | | | to λ − adjusted log10 |
| | | | | | | | (sp) <− 5, hence d = 0. |

To convey the effects of various components of the method, each Set 1, 2 and 3 of FIG. 6 and Table 3 was deliberately constructed to have very similar qualitative patterns of intensity vs. source. Yet, the examples are different in overall confidence of selective expression as determined by the method. In particular, each Set has the same source set (size n=15) and, moreover, exactly the same separation ratio (τ=0.67) before any adjustments are made for baselines. Hence, these sets have by design exactly the same traditional Dixon significance probability before baseline adjustment. Table 4 columns display, respectively: the Set identification number corresponding to FIG. 6; whether a baseline adjustment was used in the discordancy computation (equation 7); baseline adjustment factor λ (equation 8), gap (equation 3), τ (equation 4 if no baseline adjustment, otherwise equation 7), discordancy significance probability log₁₀sp (equation 6 or 10), decision function d (equation 13), and comments. Equation 9, which employs source qualities from Table 3, is used for the baseline estimates \hat{x}_{baseline} in equation 8. The equation 8 sigmoidal parameters are b=10 and c=0.8. The parameter values in the decision function (equations 11-13) are α=β=γ=1.5, δ=0.3, gₜₕᵣₑₛₕ=0.25, log₁₀(sp)ₜₕᵣₑₛₕ=−5, and log₁₀(sp)_{inf}=−20. The effects of adjusting significance probability for baseline can be seen in Table 4 by comparing each Set's case b against its respective case a, which is unadjusted for baseline. Example 3b is the only one in which significance probability is non-negligibly changed by baseline adjustment. This can be appreciated by observing the effects of baseline on λ, hence on τ, when compared against the case 1a τ. Sets 2 and 3, however, have markedly smaller gaps than does Set 1. These diminutive gaps are responsible for the decision function values for Sets 2 and 3 being much smaller than for Set 1 even though the discordancy statistical significance probabilities (with or without baseline adjustments) are not changed much. The exception is case 3a, which has an ample loss of significance probability due to baseline adjustment. Though the 3b gap is the same as 3a, 3b's decision function is zero because baseline adjustment of its statistical significance probability has resulted in its log₁₀(sp) not meeting the minimum significance criterion log₁₀(sp)ₜₕᵣₑₛₕ=−5. Taken together, these examples illustrate how qualitatively similar intensity vs. source patterns can have different overall confidence of selective expression (indicated by the decision function values), depending on the baseline of the data and the size of the gap, even when the expression patterns have essentially identical unadjusted traditional discordancy significance probabilities. By analyzing these examples, it can be seen how the qualitatively stronger confidence of selective expression of Set 1 as compared to Sets 2 and 3 (which is informally conveyed in FIG. 6) is quantitated through the decision function of the selective expression method applied to the data.

### EXAMPLE 2

### Selective Expression Detection in Gene Expression Data

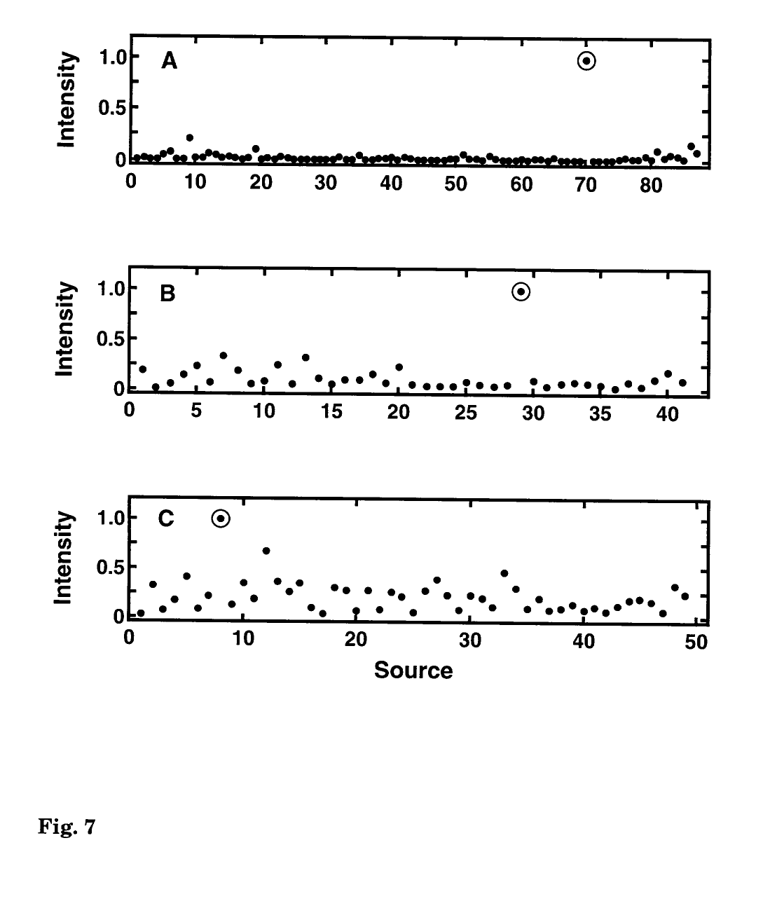

To convey the appearances of stereotypical selective expression patterns in real gene expression data, intensity vs. source plots of some actual examples of algorithmically identified Extremely Strong, Strong, and Weak overall confidence selective gene expression are shown in FIG. 7, panels A, B, and C, respectively. Shown are intensity (abundance) vs. source (library) plots for three actual assemblies from a database of real sources and assembly abundances. Assembly A has a extremely strong overall confidence of selective expression (decision function d=1.0). Assembly B has a strong overall confidence of selective expression (d=0.75). Assembly C has weak overall confidence of selective expression (d=0.31). Summarized algorithmic calculations corresponding to these examples are displayed in Table 5. The columns are similar to those in Table 4. In these particular real examples, baseline adjustment has no effect since the baselines are well below 0.8. Hence, the discordancy statistical significance probabilities are the same as the unadjusted statistical significances.

It is easily determined visually from the plots in FIG. 7 that the τ are decreasing from example A to C, with the larger decrease being from B to C. The corresponding τ are actually {0.78, 0.67, 0.35}, which agrees with this qualitative visual observation. That the discordancy statistical significance probabilities increase so dramatically with this series of τ values is due to the considerable size of the n involved, {87, 41, 49}, respectively. The marked difference in log₁₀(sp) between A and B is much more due to the difference in n than in τ. However, the substantial difference in log₁₀(sp) between B and C is due to the difference in τ more than the difference in n. These quantitations are not surprising given equation 6. Clearly, A exhibits maximum confidence as can be seen visually in FIG. 7 and quantitatively in Table 5. That the d for C is half that for B is due to both the gap and the log₁₀(sp) in combination being weaker in C than B.

TABLE 5
Selective Expression in Gene Expression Data

| | | | | | | | | Overall |
|---|---|---|---|---|---|---|---|---|
| | | | | | | log₁₀ | | Confidence |
| Set | n | ^{\hat{X}}baseline | λ | gap | τ | (sp_{adj.}) | d | in S. E. |
| A | 8 | 0.03 | 1.0 | 0.78 | 0.78 | −56.0 | 1.0 | Very Strong |
| | 7 | | | | | | | |
| B | 4 | 0.10 | 1.0 | 0.66 | 0.67 | −18.8 | 0.7 | Strong |
| | 1 | | | | | | | |
| C | 4 | 0.20 | 1.0 | 0.34 | 0.34 | −8.5 | 0.3 | Weak |
| | 7 | | | | | | | |

While it is useful for better understanding the data to dissect the various relative contributions of the ingredients of the selective expression algorithm as done above, the real power of the decision function d, is its utility in qualitatively ranking overall confidence in selective expression patterns in large scale data in a way that is not only easily automated, but objective and consistent.

References

All publications from the scientific literature cited in this specification are herein incorporated by reference as though fully set forth.

[1] R. J. Britten and D. E. Kohen, “Repeated Sequences in DNA.,” Science, vol. 161, pp. 529-540, 1968.

[2] G. A. Galau, W. H. Klein, R. J. Britten, and E. H. Davidson, “Significance of Rare mRNA Sequences in Liver,” Archives of Biochemistry and Biophysics, vol. 179, pp. 584-599, 1977.

[3] B. D. Hames and S. J. Higgins, “Nucleic Acid Hybridisation—A Practical Approach,” in The Practical Approach Series. Oxford, UK: IRL Press Limited, 1985, pp. 245.

[4] S. Patanjali, S. Parimoo, and S. M. Weissman, “Construction of a Uniform-Abundance (Normalized) cDNA Library,” Proceedings of the National Academy of Sciences USA, vol. 88, pp. 1943-1947, 1991.

[5] M. D. Adams, “Expressed Sequence Tags as Tools for Physiology and Genomics,” in Automated DNA Sequencing and Analysis, M. D. Adams, C. Fields, and J. C. Venter, Eds. London: Academic Press Ltd., 1994, pp. 71-80.

[6] M. Singer and P. Berg, Genes & Genomes. Mill Valley, Calif.: University Science Books, 1991.

[7] M. R. Wilkins, K. L. Williams, R. D. Appel, and D. F. Hochstrasser, “Proteome Research: New Frontiers in Functional Genomics,” in Principles and Practice. Berlin: Springer-Verlag, 1997, pp. 243.

[8] H. Lodish, D. Baltimore, A. Berk, S. L. Zipursky, P. Matsudaira, and J. Darnell, Molecular Cell Biology, Third Edition ed. New York: Scientific American Books/W. H. Freeman and Co., 1995.

[9] M. D. Adams, C. Fields, and J. C. Venter, “Automated DNA Sequencing and Analysis,” London: Academic Press Ltd., 1994, pp. 368.

[10] N. L. Anderson, J. -P. Hofmann, A. Gemmell, and J. Taylor, “Global Approaches to Quantitative Analysis of Gene-Expression Patterns Observed by Two-Dimensional Gel Electrophoresis,” Clinical Chemistry, vol. 30, pp. 2031-2036, 1984.

[11] L. Anderson and J. Seilhamer, “A Comparison of Selected mRNA and Protein Abundances in Human Liver,” Electrophoresis, vol. 18, pp. 533-537, 1997.

[12] C. Burks, M. L. Engle, S. Forrest, R. J. Parsons, C. A. Soderlund, and P. E. Stolorz, “Stochastic Optimization Tools for Genomic Sequence Assembly,” in Automated DNA Sequencing and Analysis, M. D. Adams, C. Fields, and J. C. Venter, Eds. London: Academic Press Ltd., 1994, pp. 250-259.

[13] E. W. Myers, “Advances in Sequence Assembly,” in Automated DNA Sequencing and Analysis, M. D. Adams, C. Fields, and J. C. Venter, Eds. London: Academic Press Ltd., 1994, pp. 231-248.

[14] B. R. Herbert, J. -C. Sanchex, and L. Bini, “Two-Dimensional Electrophoresis: The State of the Art and Future Directions,” in Proteome Research: New Frontiers in Functional Genomics, M. R. Wilkins, K. L. Williams, R. D. Appel, and D. F. Hochstrasser, Eds. Berlin: Springer-Verlag, 1997, pp. 13-33.

[15] J. Bunge and M. Fitzpatrick, “Estimating the Number of Species: A Review,” Journal of American Statistical Association, vol. 88, pp. 364-373, 1993.

[16] W. A. Lewins and D. N. Joanes, “Bayesian Estimation of the Number of Species,” Biometrics, vol. 40, pp. 323-328, 1984.

[17] V. Barnett and T. Lewis, Outliers in Statistical Data: Chichester & New York, 1978.

[18] G. L. Tietjen, “The Analysis and Detection of Outliers,” in Goodness-of-Fit Techniques, vol. 68, Statistics, Textbooks and Monographs, R. B. D'Agostino and M. A. Stephens, Eds. New York: Marcel Dekker, Inc., 1986, pp. 497-521.

[19] D. M. Hawkins, Identification of Outliers. London & New York: Chapman & Hall, 1980.

[20] R. B. D'Agostino and M. A. Stephens, “Goodness-of-Fit Techniques,” in Statistics, Textbooks and Monographs, vol. 68. New York: Marcel Dekker, Inc., 1986.

[21] L. Sachs, Applied Statistics—A Handbook of Techniques, 2nd ed. New York: Springer-Verlag, 1982.

It is contemplated that other statistical tests of outlier discordancy may be used in place of the Dixon test [17] in Steps (c), (d), and (f). Further, the decision function may have a mathematical form different than equation (13) which may be used in Steps (f) and (g). The properties of a decision function d are what matters more than the particular mathematical form (e.g, equation (13)) that is chosen: Decision function d near 0 is interpreted as very weak overall confidence, while d near 1 is very strong overall confidence in selective expression. d is designed to capture the following notions of confidence:

| | | scaled gap g | scaled gap g |
|---|---|---|---|
| | | weak | strong |
| | | (g˜0) | (g˜1) |
| scaled sig. | weak | weak | strong |
| prob. s | (s˜0) | (d˜0) | (d˜1) |
| scaled sig. | strong | moderate | strong |
| prob. s | (s˜1) | (d˜1) | (d˜1) |

d is (1) strong when both the baseline adjusted log₁₀sp and the gap are strong (i.e., both s and g are near 1); (2) weak when both the loglosp and the gap are weak (i.e. s and g near 0); (3) moderate when the log₁₀sp is strong but the gap is weak; (4) but strong nonetheless when the gap is strong yet the loglosp is weak. Notions (3) and (4) make sense because both the log₁₀sp and the gap that are considered in the decision function confidence assessment are stronger their respective minimum thresholds. Either log₁₀sp or gap weaker than their respective minimum thresholds is not selective expression, and immediately d=0 in such cases. There is no a priori requirement that d be symmetrical with respect to g and s. In fact, in practice, an asymmetry is preferred that gives d near 1 for large gaps as long as log₁₀sp is stronger than a threshold value.

It will be apparent to those skilled in the art that various modifications can be made to the present method without departing from the scope or spirit of the invention, and it is intended that the present invention cover modifications and variations of the method provided they come within the scope of the appended claims and their equivalents.

1. A method of identifying selectively expressed values in intensity data comprising analyzing statistical discordancy and gap criterion in a decision function wherein the decision function provides an overall confidence of above- or below-baseline exceptional intensity identification.

2. The method of claim 1 wherein the statistical discordancy is adjusted for baseline intensity levels.

3. A method of identifying exceptional values in intensity data comprising:
(a) selecting intensity values from intensity data sources, wherein confidence in source quality exceeds a predetermined minimum threshold;
(b) determining if the number of selected intensities exceeds a predetermined minimum;
(c) applying a statistical discordancy test to identify statistically significant exceptional intensity values;
(d) determining a gap between the largest and another intensity by applying a minimum intensity gap criterion to the results of the statistical discordancy test;
(e) applying a decision function to the discordancy statistical significance and the gap to determine an overall confidence of exceptional intensity;
(f) identifying the degree of overall confidence of exceptional intensity; and
(g) displaying the results of step (f) on an output device.

4. The method of claim 3 wherein the statistical discordancy test results of step (c) are adjusted according to the difference between a baseline position and a maximum allowed intensity to achieve a baseline adjusted statistical significance.

5. The method of claim 3 wherein the gap is determined between the largest and the next-to largest intensity.

6. The method of claim 1 or 3 wherein the intensity data is from tissue or cDNA library sources.

7. The method of claim 1 or 3 wherein the intensity data is from human sources.

8. The method of claim 1 or 3 wherein the intensity data is from non-human sources.

9. The method of claim 8 wherein the intensity data is from animal, plant, viral, bacterial, or microbial sources.

10. The method of claim 1 or 3 wherein the intensity data is from genomic sequencing, EST sequencing, microarray DNA hybridization, macromolecular gridding, compound assays, molecular screening assays, patient diagnostic or toxicological data sources.

11. The method of claim 3 wherein the source quality confidence is based on trust, reliability, knowledge of error or relevance.

12. The method of claim 3 wherein the intensity baseline position is determined by a source quality weighted average of the intensities.

13. The method of claim 3 further comprising the step of characterizing the selectively expressed genes or gene products.

14. A method of detecting selective expression of genes or gene products comprising:
(a) selecting intensity values from gene product data sources, wherein confidence in source quality exceeds a predetermined minimum threshold;
(b) determining if the number of selected intensities exceeds a predetermined minimum;
(c) applying a statistical discordancy test to identify statistically significant exceptional intensity values;
(d) determining a gap between the largest and another intensity by applying a minimum intensity gap criterion to the results of the statistical discordancy test;
(e) applying a decision function to the discordancy statistical significance and the gap to determine an overall confidence of selective expression;
(f) identifying the degree of overall confidence of selective expression; and
(g) displaying the results of step (f) on an output device.

15. The method of claim 14 wherein the statistical discordancy test results of step (c) are adjusted according to the difference between a baseline position and a maximum allowed intensity to achieve a baseline adjusted statistical significance.

16. The method of claim 14 wherein the source quality confidence is based on trust, reliability, knowledge of error or relevance.

17. The method of claim 14 wherein the baseline position is determined by a source quality weighted average of the intensities.

18. The method of claim 14 further comprising the step of characterizing the selectively expressed genes or gene products.

19. A computer system for identifying selectively expressed values in intensity data comprising means for analyzing statistical discordancy and gap criterion in a decision function wherein the decision function provides an overall confidence of above- or below-baseline exceptional intensity identification.

20. A computer system for identifying exceptional values in intensity data comprising:
(a) means for selecting intensity values from intensity data sources, wherein confidence in source quality exceeds a predetermined minimum threshold;
(b) means for determining if the number of selected intensities exceeds a predetermined minimum;
(c) means for applying a statistical discordancy test to identify statistically significant exceptional intensity values;
(d) means for determining a gap between the largest and another intensity by applying a minimum intensity gap criterion to the results of the statistical discordancy test;
(e) means for applying a decision function to the discordancy statistical significance and the gap to determine an overall confidence of exceptional intensity;
(f) means for identifying the degree of overall confidence of exceptional intensity; and
(g) means for displaying the results of step (f) on an output device.

21. A computer readable medium containing program instructions for identifying selectively expressed values in intensity data comprising analyzing statistical discordancy and gap criterion in a decision function wherein the decision function provides an overall confidence of above- or below-baseline exceptional intensity identification.

22. A computer readable medium containing program instructions for identifying exceptional values in intensity data, the program instructions comprising:
(a) selecting intensity values from intensity data sources, wherein confidence in source quality exceeds a predetermined minimum threshold;
(b) determining if the number of selected intensities exceeds a predetermined minimum;
(c) applying a statistical discordancy test to identify statistically significant exceptional intensity values;
(d) determining a gap between the largest and another intensity by applying a minimum intensity gap criterion to the results of the statistical discordancy test;
(e) applying a decision function to the discordancy statistical significance and the gap to determine an overall confidence of exceptional intensity;
(f) identifying the degree of overall confidence of exceptional intensity; and
(g) displaying the results of step (f) on an output device.
